(19) United States
(12) Patent Application Publication
(10) Pub. No.: US 2002/0095129 A1
(43) Pub. Date: Jul. 18, 2002

(54) Body fluid sealing gaskets for personal care products

(76) Inventors: S. Scott Friderich, Alpharetta, GA (US); Jay Sheldon Shultz, Roswell, GA (US)

Correspondence Address: Pauley Petersen Kinne & Erickson, Suite 365, 2800 W. Higgins Road, Hoffman Estates, IL, 60195, US

(21) Appl. No.: 10/024,634

(22) Filed: Dec. 17, 2001

Related U.S. Application Data
(60) Provisional application No. 60/257,765, filed on Dec. 21, 2000.

(51) Int. Cl. A61F 13/15

(52) U.S. Cl. 604/385.28

(57) ABSTRACT

A containment flap for an absorbent article, such as an incontinence garment, is made from a breathable liquid barrier material. The barrier material may further be laminated to additional materials. The flap desirably has elasticity in the long axis and extensibility of the flap with a low modulus of elasticity in its transverse direction. The long axis tension of this flap has a force vector normalized to the transverse direction when the flap is placed in curvature over the body of a wearer, thereby providing a force for extending the flap in the transverse direction to maintain contact with the body of the wearer when the garment begins to sag, such as may happen due to gravity when the garment is loaded with absorbed bodily fluids.

### BACKGROUND OF THE INVENTION

Pant-like absorbent garments, such as diapers and training pants, typically include a pair of leg openings having an elastic portion around each leg opening, and a waist opening having an elastic portion as well. The elastic portions are intended to fit snugly around a wearer's legs to prevent leakage from the garment, yet leakage often persists.

A number of different approaches have been taken to reduce or eliminate leakage from absorbent garments. For example, physical barriers, such as elasticized containment flaps, have been incorporated into such absorbent garments. The amount and configuration of absorbent material in the zone of the absorbent garment in which liquid surges typically occur (sometimes referred to as a target zone) have also been modified.

A further approach to decreasing body fluid leakage is to increase tension of the elastic portions around each leg opening and the waist opening. The increased tension is often effective, but just as often results in an undesirable red marking on a wearer's skin due to increased pressure on the wearer's skin.

The use of containment flaps has, in the past, been somewhat limited because the flaps are either not good liquid barriers, such as in previously utilized carded web fabrics; not breathable; or of a finite width in their transverse direction; or various combinations of these shortcomings. Lack of a good liquid barrier is an obvious shortcoming to the purpose of the flaps. Breathability will contribute to overall user comfort but should not be had at the expense of good liquid barrier properties, such as in previously disclosed flaps using pierced film material. The lack of transverse extension of the flap material will mean that, as the absorbent garment becomes loaded with absorbed bodily wastes and sags or droops due to gravity, the gasket will pull away from the wearer's body, sometimes called “a loss of vertical fit”, thereby providing an unwanted leakage path for solids or liquids to the exterior of the garment. Providing “oversized” gaskets to try and accommodate a range of sagging is known in the art but results in an undesirable increase of fabric usage from both economical and comfort standpoints. One type of breathable stretchable thin film laminate is disclosed in U.S. Pat. Nos. 5,695,868 and 5,855,999 to McCormack, of common ownership herewith, and which are hereby incorporated by reference in their entirety. To applicants's knowledge this material has not previously be used in conjunction with gaskets per the teachings of the present invention.

There is a need or desire for gaskets or containment flaps in absorbent garments that seal fluid within the absorbent garments while remaining breathable, and which may add further advantages such as adjustment in the transverse direction to the level of sagging in a loaded garment in order to maintain gasketing. There is a further need for economical materials which can provide such flaps.

### SUMMARY OF THE INVENTION

The present invention is directed to improved materials and constructions of gaskets in pant-like absorbent garments. The material used for the gaskets, or flaps, is selected to be both breathable and a barrier material to liquids, such as a microporous film. In an exemplary garment, transversely extensible gaskets may provide greater leakage protection by maintaining contact with the body of the wearer by extending in the transverse direction when the garment sags away from the body, and further provide a true liquid barrier while remaining breathable.

Aspects of the present invention may include absorbent articles with gaskets having a breathable and transversely extendible film. The film may then be covered with nonwoven facings for a softer feel. Microporous films suitable for use with the present invention should have a light ounce per square yard (osy) basis weight range so as to easily conform to the body of the wearer and be easily gathered by elastic treatments in the flap if desired. Microporous films may be incorporated with nonwoven webs, such as spunbond facing material, or other components to create laminates. In these cases the non-film component of the laminate should have a light basis weight also. For example, the spunbond facing is desirably 0.5 osy or less in a total film/nonwoven basis weight under 2.0 osy, in order to maintain excellent gatherability of the material. Lighter films will consume less polymer in the making and therefore be more economical and environmentally friendly. As set forth in greater detail below, the microporous film may be laminated to other materials for various esthetic or functional purposes, or both. Microporous film suitable for certain embodiments of the present invention is further unidirectionally stretched during manufacture, leading to greater extensibility in one direction, which will become the transverse direction of the gasket. The exact level or extent of transverse extensibility may vary according to the usage to which the gasket is put as will be understood upon a full understanding of the present invention. Microporous film is, by definition herein, a breathable, liquid water barrier material, although the degree of breathability suitable for certain embodiments of the present invention may vary according to selected usage.

In one embodiment the barrier material comprises a breathable stretchable thin film laminate, one type of which is disclosed in the aforementioned U.S. Pat. Nos. 5,695,868 and 5,855,999 to McCormack. Breathable stretchable thin film laminates generally comprise a microporous film which is breathable and also a barrier material to the passage of fluid. The film is laminated to a nonwoven web having the desired comfort and functional properties without compromise to the structural integrity of the films.

A disposable garment according to the present invention includes gasketing typically provided by elasticized flap portions which are connected to the interior of the garment along the lower part of each leg opening. Throughout use, the elasticized flap portions fit snugly against the wearer and effectively block most spillage of waste material from the leg openings.

It will be appreciated that when flap portions are used for the leak guards, a separate manufacturing step can be required to attach the flap material to the garment. Generally, the flaps have been joined via seams. During active use, some separation at the seams can occur, resulting in failure of the flaps to serve as effective leak guards. Providing a seam which is both leakproof and durable has been challenging, and has added to manufacturing costs. To solve this problem, seamless leak guards were disclosed in co-pending U.S. application Ser. No. 09/290,414, of common ownership herewith. The present invention is also applicable to the integral, or seamless, method of providing gasketing.

As described in the co-pending application, instead of using flaps, seamless leak guards may be provided by extending the breathable, liquid-impermeable outer cover layer substantially beyond the absorbent layer on both sides, and to a higher location on the garment and on the wearer. The outer cover extensions on both sides can be reinforced at their edges by elastic leg bands which pull the outer cover extensions upward and away from the absorbent layer, and against the wearer's body. The lateral extensions of the outer cover material, combined with the upward pulling of the elastic leg bands, may provide the garment with seamless leak guards not requiring separately attached flaps.

### BRIEF DESCRIPTION OF THE DRAWINGS

FIG. 1 is a vector diagram of tension and pressure at a point along a curved surface;

FIG. 2 is a front perspective view of a known absorbent garment given as an environment of the present invention;

FIG. 3 is a top plan view of an absorbent garment assembly;

FIG. 4 is a simplified top plan view of the garment indicating tensioning of the flaps in their long axis and extensibility of the flaps in their transverse axis;

FIG. 5 is a cross-sectional view of the absorbent garment assembly, taken along line 5-5 in FIG. 4, and illustrating transverse extensibility of the gasket;

FIG. 6 is a schematic side view of the garment, and a wearer thereof, illustrating the extensible flap material gasketing in response to a loading of the garment with absorbed fluids.

FIG. 7 is a schematic top view of another embodiment of the invention wherein the flap is integral with the chassis.

FIG. 8 is a schematic cross section of a part of a breathable extensible laminate suitable for use with the present invention.

### DEFINITIONS

Within the context of this specification, each term or phrase below will include the following meaning or meanings.

“Article” refers to a garment or other end-use article of manufacture, including but not limited to absorbent articles such as diapers; training pants; swim wear; absorbent underpants; adult incontinence articles; feminine hygiene articles; and medical garments and wraps.

“Attached” can refer to either an integral part or a part joined by a separate joining process.

A “barrier” or “impervious” material is a material with no measurable transmission of a selected substance through that material over the expected term of use of the material.

“Bicomponent” nonwoven filaments are known in the art generally as thermoplastic filaments which employ at least two different polymers combined together in a heterogeneous fashion. Instead of being homogeneously blended, two polymers may, for instance, be combined in a side-by-side configuration, so that a first side of a filament is composed of a first polymer “A” and a second side of the filament is composed of a second polymer “B.” Alternatively, the polymers may be combined in a sheath-core configuration, so that an outer sheath layer of a filament is composed of a first polymer “A,” and the inner core is composed of a second polymer “B.” Other heterogeneous configurations are also possible.

“Bonded” refers to the joining, adhering, connecting, attaching, or the like, of two elements. Two elements will be considered to be bonded together when they are bonded directly to one another or indirectly to one another, such as when each is directly bonded to intermediate elements.

The term “breathable” refers to a material which is permeable to water vapor having a minimum WVTR of sufficient functionality for the comfort of the wearer. The WVTR of a fabric is water vapor transmission rate which, in one aspect, gives an indication of how comfortable a fabric would be to wear. WVTR is reported in grams/square meter/day and can be measured as described herein below.

The term “cloth” includes, but is not limited to, a fabric made of fibrous material, commonly a woven fabric of, for example, cotton. Furthermore, the term “cloth” shall also include all nonwoven materials exhibiting a cloth-like feel.

“Connected” refers to the joining, adhering, bonding, attaching, or the like, of two elements. Two elements will be considered to be connected together when they are connected directly to one another or indirectly to one another, such as when each is directly connected to intermediate elements.

“Disposable” refers to articles which are designed to be discarded after a limited use rather than being laundered or otherwise restored for reuse.

“Disposed,” “disposed on,” and variations thereof are intended to mean that one element can be integral with another element, or that one element can be a separate structure bonded to or placed with or placed near another element.

“Elastic,” “elasticized,” “elastomeric,” and “elasticity” mean that property of a material or composite by virtue of which it tends to recover its original size and shape after removal of a force causing a deformation. “Extensible” implies little or no recovery of the original size or shape.

“Extensible” or “extendible” implies extension under a deformation force with little or no recovery of the original size or shape after the deformation force is removed. A “low modulus of elasticity” with respect to an extensible material implies that little force is required to extend the material and is not meant to imply that the extensible material exhibits elasticity.

The term “film” refers to a thermoplastic film made using a film extrusion process, such as a cast film or blown film extrusion process. This term includes films rendered microporous by mixing polymer with filler, forming a film from the mixture, and stretching the film.

“Gaskets”, also called “cuffs” or “containment flaps”, in some instances, are structures within, or on, the personal care product serving as barriers to the escape of bodily exudates. The terms “gaskets”, “flaps” and “containment flaps” will be used interchangeably throughout the application.

“Integral” or “integrally” is used to refer to various portions of a single unitary element rather than separate structures bonded to or placed with or placed near one another.

“Layer” when used in the singular can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, may spread or be transported parallel to the plane of the liquid impermeable layer or laminate, but this is not considered to be within the meaning of “liquid impermeable” when used herein.

“Longitudinal” and “transverse” have their customary meaning, as indicated by the longitudinal and transverse directions depicted in FIG. 4 at arrows 62 and 66, respectively. The longitudinal, or long, axis lies in the plane of the article and is generally parallel to a vertical plane that bisects a standing wearer into left and right body halves, when the article is worn. The transverse axis lies in the plane of the article generally perpendicular to the longitudinal axis. The article and its parts, although illustrated as longer in the longitudinal direction than in the transverse direction, need not be so.

“Machine direction,” or MD, refers to the length of a fabric in the direction in which it is produced, as opposed to “cross direction,” or CD, which refers to the width of a fabric in a direction generally perpendicular to the machine direction.

“Meltblown fiber” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are desirably substantially continuous in length.

“Member” when used in the singular can have the dual meaning of a single element or a plurality of elements.

The term “microporous” refers to films having voids separated by thin polymer membranes and films having micropores passing through the films. The voids or micropores may be formed when a mixture of polymer and filler is extruded into a film and the film is stretched, e.g., transversely in the machine direction. Microporous films tend to have water vapor transmission due to molecular diffusion of water vapor through the membranes or micropores, but substantially block the passage of aqueous liquids.

As used herein, the term “necked material” refers to any material which has been drawn in at least one dimension, (e.g. lengthwise), reducing the transverse dimension, (e.g. width), such that when the drawing force is removed, the material can be pulled back, or relax, to, or near, its original width. The necked material typically has a higher basis weight per unit area than the un-necked material. When the necked material returns to its original un-necked width, it should have about the same basis weight as the un-necked material. This differs from stretching/orienting a material layer, such as a film, during which the layer is thinned and the basis weight is permanently reduced.

The term “nonwoven fabric” or “nonwoven web” means a web having a structure of individual fibers or threads which are interlaid, but not in a regular or identifiable manner as in a knitted fabric. Nonwoven fabrics or webs have been formed from many processes such as, for example, meltblowing processes, spunbonding processes, air-laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91).

“Permanently bonded” refers to the joining, adhering, connecting, attaching, or the like, of two elements of an absorbent garment such that the elements tend to be and remain bonded during normal use conditions of the absorbent garment.

The term “personal care absorbent product” includes without limitation diapers, training pants, swim wear, absorbent underpants, baby wipes, adult incontinence products, and feminine hygiene products.

Words of degree, such as “about”, “substantially”, and the like are used herein in the sense of “at, or nearly at, when given the manufacturing and material tolerances inherent in the stated circumstances” and are used to prevent the unscrupulous infringer from unfairly taking advantage of the invention disclosure where exact or absolute figures are stated as an aid to understanding the invention.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and about 10.

As used herein, the term “substantially continuous fibers” refers to fibers, including without limitation, spunbond and meltblown fibers, which are not cut from their original length prior to being formed into a nonwoven web or fabric. Substantially continuous fibers may have average lengths ranging from greater than about 15 centimeters to more than one meter, and up to the length of the web or fabric being formed. The definition of “substantially continuous fibers” includes fibers which are not cut prior to being formed into a nonwoven web or fabric, but which are later cut when the nonwoven web or fabric is cut, and fibers which are substantially linear or crimped.

“Thermoplastic” describes a material that softens when exposed to heat and which substantially returns to a nonsoftened condition when cooled to room temperature.

A “transversely” stretchable, or extendible, material is one which extends more easily along a first axis than along a second axis. “Transversely” extendible is not necessarily meant to imply that there is no extendibility in the second axis.

These terms may be defined with additional language in the remaining portions of the specification.

### DETAILED DESCRIPTION OF THE PRESENTLY PREFERRED EMBODIMENTS

Aspects of the present invention generally include gaskets, and materials having a breathable, liquid barrier, film with transverse extensibility. Microporous film is a good choice for gasket material in that it is breathable to contribute to the comfort of the wearer, while being an absolute barrier, for practical purposes, to the passage of aqueous liquids and, of course solids, thereby performing the function of preventing leakage of bodily exudates beyond the boundaries of an absorbent garment or article of the gasket. Microporous films desirably used with the present invention should have a light osy basis weight range so as to easily conform to the body of the wearer and be easily gathered by elastic treatments in the flap if desired in selected embodiments of the gasket. Lighter films will also consume less polymer in the making and therefore be more economical and environmentally friendly. Further, certain lighter films may exhibit good flexibility and gatherability which can be desirable traits for the gaskets in certain garments according to the present invention. Certain microporous films may be easily laminated to other materials for various esthetic and functional purposes, such as comfortable skin feel, or hand, and improved liquid transfer properties away from the barrier area. Microporous film suitable for certain embodiments of the present invention where transverse extensibility of the flap is desired may be unidirectionally stretched during manufacture, leading to greater extensibility in one direction, which will usually become the transverse direction of the gasket. The exact level or extent of transverse extensibility may vary according to the usage to which the gasket is put, as will be understood upon a full understanding of the present invention. Microporous film is, by definition herein, a breathable, liquid water barrier, material, although the degree of breathability suitable for certain embodiments of the present invention may vary according to selected usage or product designation.

By way of explanation of the present invention, particular embodiments having many of the characteristics of a desirable gasket material according to the present invention will be set forth, including light weight, transverse extensibility, lamination to another material to improve feel and function, breathability and barrier function to liquids, but are not to be taken as exclusive examples or limiting to the invention.

Referencing FIG. 1, in relation to an exemplary embodiment of the present invention, it is well known that pressure exerted from elastic tension at a given body contact point is proportional to the curvature at the point as well as to the amount of tension, as demonstrated by the LaPlace equation:

P_{g}=σ₁R₁ (1)

where P_{g} is the normal force or gasket pressure, R₁ is the radius of curvature along a wearer's body 5, and al is the tension in the tangential direction (see FIG. 1). Thus, it can be appreciated that pressure P_{g} is generated under a given tension σ₁. P_{g} is a force aligned in the transverse direction of the containment flap providing a force to extend the flap as further explained below.

In pant-like absorbent garments having elasticized leg openings and/or an elasticized waist opening, the elastic tension σ₁ should be high enough so that sufficient pressure P_{g} is exerted at all points around the perimeter of the opening, to seal the garment against the wearer's body. This force in the present invention should also be in an amount to easily achieve full extension of the flap material towards the wearer.

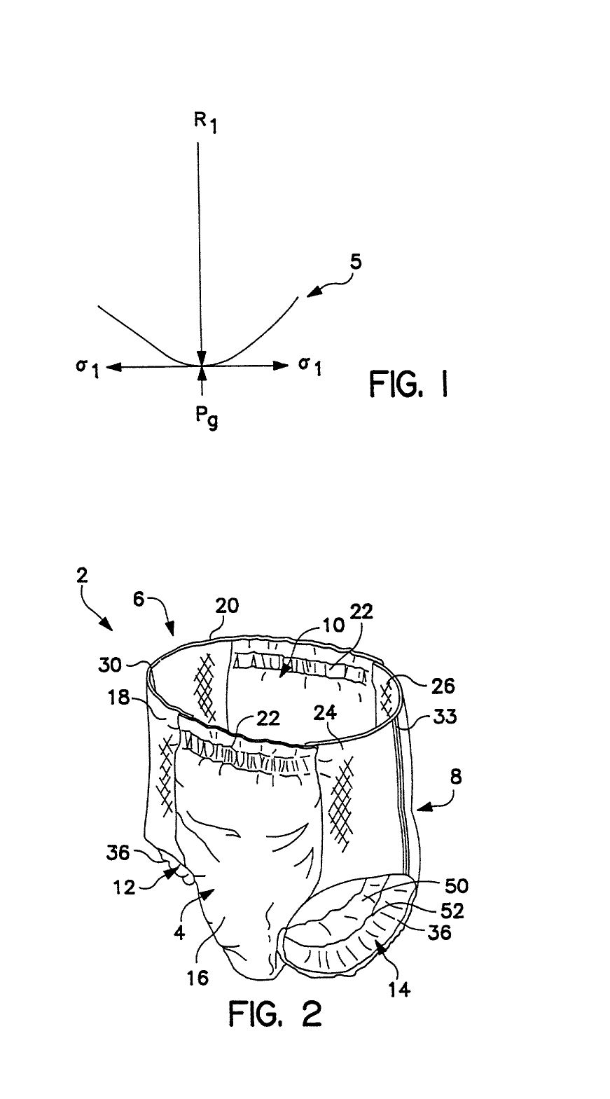

Referring to FIG. 2, a conventional pant-like absorbent garment 2 for use in conjunction with the present invention includes a waste containment section 4 and two side portions 6 and 8 defining a waist opening 10 and a pair of leg openings 12 and 14. The side portion 6 includes stretchable panels 18 and 20 joined together at seam 30. The side portion 8 includes stretchable panels 24 and 26 joined together at seam 33. Seams 30 and 33 extend longitudinally from the waist opening 10 to the leg openings 12 and 14 of the garment 2.

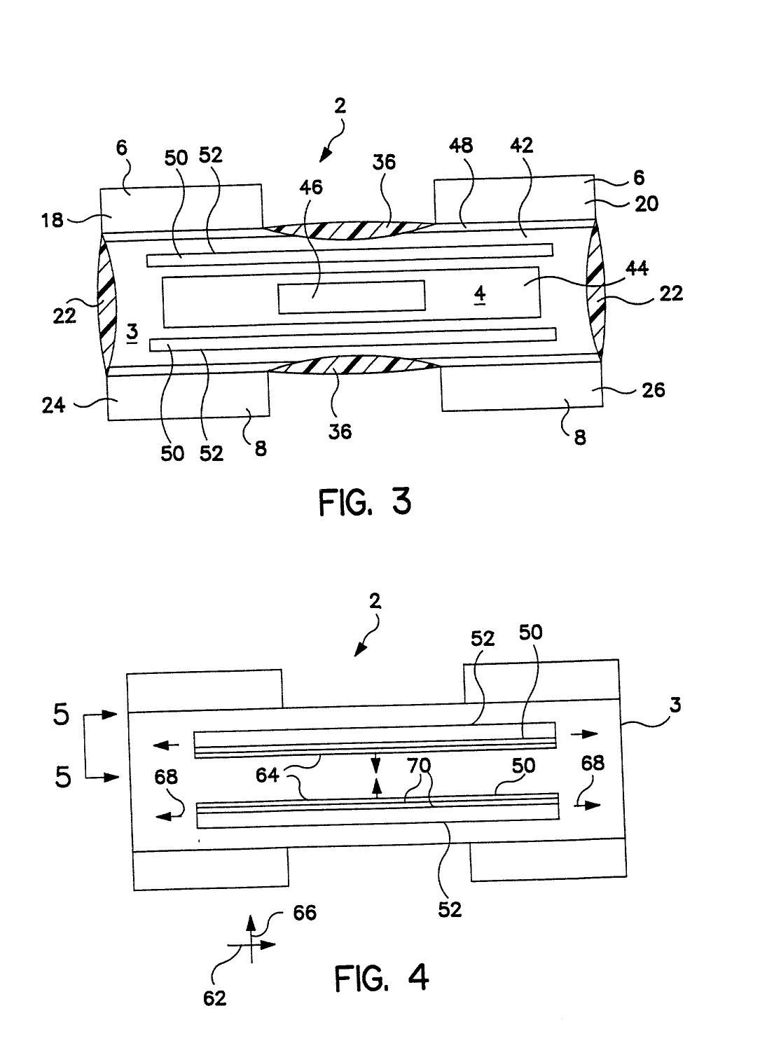

The waste containment section 4 includes multiple layers, as shown in FIG. 3, including, for instance, a liquid-permeable body side liner 42, an absorbent core layer 44, a surge layer 46, and a liquid-impermeable outer cover 48 which faces away from the wearer. The waste containment section 4 includes waist elastics 22 on the front and back of the garment 2. The leg openings 12 and 14 also include leg elastics 36 which extend substantially around the portion of the leg openings defined by the waste containment section 4.

The stretchable side portions 6 and 8 can be constructed of conventional woven or nonwoven materials, formed from a wide variety of elastic and stretchable polymers. Suitable polymers include without limitation block copolymers of polystyrene, polyisoprene and polybutadiene; copolymers of ethylene, natural rubbers and urethanes; and combinations of the foregoing. Particularly suitable are styrene-butadiene block copolymers which have been sold by Shell Chemical Co. under the trade name KRATON®. Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof. Also suitable are coextruded composites of the foregoing, and elastomeric staple integrated composites where staple fibers of polypropylene, polyester, cotton and other materials are integrated into an elastomeric meltblown web. Certain elastomeric ultra-low density polymers such as single-site or metallocene-catalyzed olefin polymers and copolymers are also suitable for the side portions 6 and 8. Referencing FIGS. 2 and 3, the stretchable side portions 6 and 8 are desirably rectangular in shape, and as shown in FIG. 2, extend from the top of the waist opening 10 to the leg openings 12 and 14. The side portions 6 and 8 may also be laminates of multiple layers, and are desirably breathable to water vapor but impervious to liquids.

When an absorbent garment chassis 3, shown in FIG. 3, including all parts of the absorbent article exclusive of the flaps 50, is assembled into the absorbent garment shown in FIG. 2, the longitudinal seams 30 and 33 may be formed by conventional methods including, without limitation, ultrasonic welding, thermal bonding, adhesive bonding, stitch bonding and the like. Ultrasonic welding is a presently desirable technique. The various bonding techniques are conventional, and are neither critical nor limiting as to the present invention.

The leg elastics 36 may be attached to the outer cover 48 by a variety of techniques including adhesive bonding, ultrasonic bonding, thermal bonding, stitch bonding or other conventional techniques. Suitable adhesives include spray adhesives, hot melt adhesives, self-adhering elastomeric materials and the like. Often, the leg elastics 36 will be applied in the stretched condition to the outer cover 48, and then allowed to retract, causing gathering of the outer cover 48 when the leg elastics 36 are retracted. The leg elastics 36 desirably comprise at least two elastic bands, more desirably at least four elastic bands.

In the vicinity of the waist opening 10, the waist elastics 22 may be attached to or embedded within the garment 2. The waist elastics 22 may include single or multiple elastic bands constructed from any of the same materials as the leg elastics 36. The waist elastics 22 in the front and back of the garment 2 desirably have lengths which are nearly the same, or slightly shorter than the width of the outer cover 48. The waist elastics 22 may be attached to the outer cover 48 using the same techniques as for attaching leg elastics 36.

A wide variety of elastic materials may be employed for the leg elastics 36 and the waist elastics 22. Examples include a film or meltblown web formed using block or graft copolymers of butadiene, isoprene, styrene, ethylene-methyl acrylate, ethylene-vinyl acetate, ethylene-ethyl acrylate or blends thereof. One desirable elastomer is a block copolymer of styrene-ethylbutadiene-styrene. Polyester elastomeric materials, polyurethane elastomeric materials and polyamide elastomeric materials can be used as well. Elastomeric ultra-low density polymers such as single-site or metallocene-catalyzed olefin polymers and copolymers can also be employed. Also, the leg elastics 36 and the waist elastics 22 can be made of an activatable material applied in an unstretched condition, and activated by heat, light or moisture or radiation to cause shrinkage and elasticity.

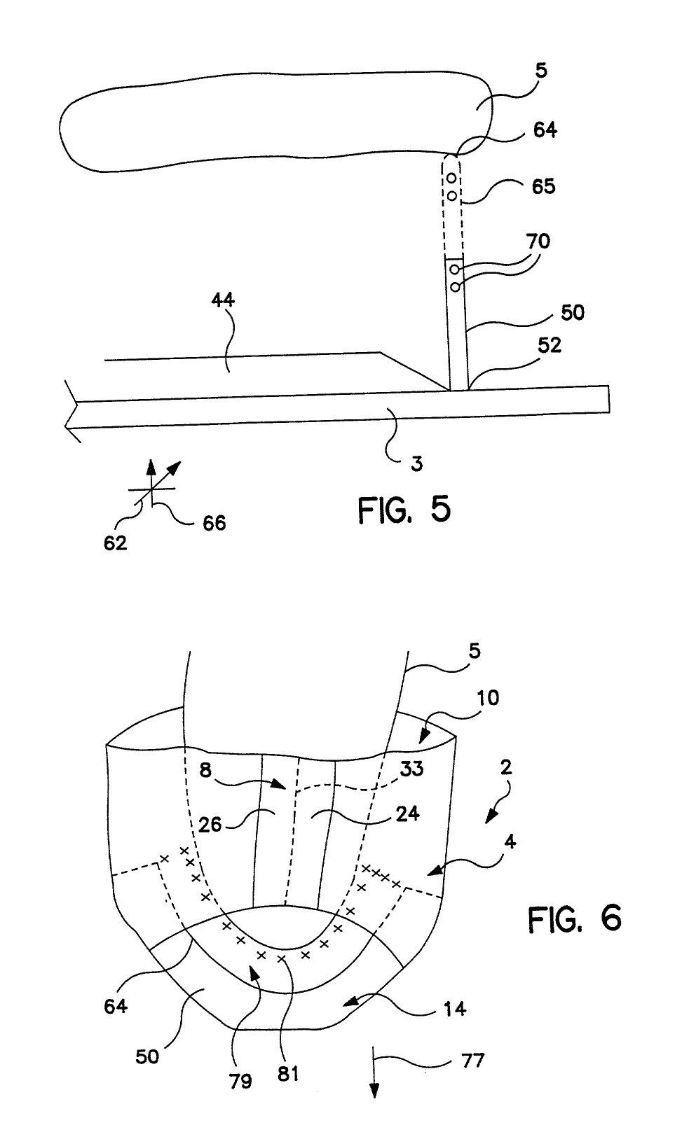

As previously indicated, the outer cover 48 may include a single layer, or may include multiple layers joined together. The outer cover 48, as shown in FIGS. 4 and 5, may include two layers, a cloth layer and a polymer layer, joined by an outer cover adhesive layer. The cloth layer of the outer cover 48 can be made from a wide variety of woven or nonwoven material, films, or a film-coated nonwoven material, including, for instance, cast or blown films of polyethylene, polypropylene, polyester or blends thereof. The cloth layer may also be a composite of a bonded carded or spunbond or meltblown material, for example, a spunbond-meltblown composite of thermoplastic material or a spunbond-meltblown-spunbond thermoplastic material, wherein the spunbond layer can provide a cloth-like texture and the meltblown layer can provide liquid impermeability. Materials of which the cloth layer can be made include nonwovens having a basis weight of about 0.4 ounces per square yard (13.6 gsm) or greater. The polymer layer of the outer cover 48 can include extruded films of polyolefin polymers or copolymers, or other thermoplastic materials.

The outer cover 48, absorbent core layer 44, surge layer 46 and body side liner 42 may also be joined together using ultrasonic bonding, thermal bonding, stitch bonding, or any of the adhesive materials described above for the attachment of the leg elastics 36 and the waist elastics 22.

The absorbent core layer 44 can, without limitation, be made of wood pulp fluff or a mixture of wood pulp fluff and a superabsorbent material, or a wood pulp fluff integrated with a thermoplastic absorbent material treated with a surfactant, or absorbent foams. Thermal binders, such as Pulpex® can be used in blends or layering with the fluff and superabsorbent material. The absorbent core layer 44 can also include a batt of meltblown synthetic fibers, a bonded carded web of synthetic or natural fibers or blends thereof, a composite of meltblown fibers and the like. The synthetic fibers can be, but are not limited to, polypropylene, polyethylene, polyester and copolymers of these or other polyolefins.

Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further superabsorbent materials include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthum gum, locust bean gum and the like. Mixtures of natural and wholly or partially synthetic superabsorbent polymers can also be useful in the present invention. Other suitable absorbent gelling materials are disclosed by Assarsson et al. in U.S. Pat. No. No. 3,901,236 issued Aug. 26, 1975. Processes for preparing synthetic absorbent gelling polymers are disclosed in U.S. Pat. No. 4,076,663 issued Feb. 28, 1978 to Masuda et al. and U.S. Pat. No. 4,286,082 issued Aug. 25, 1981 to Tsubakimoto et al.

Both the surge layer 46 and the body side liner 42 are constructed from liquid pervious materials. These layers function to transfer liquid from the wearer to the absorbent core layer 44. Suitable materials include porous woven materials, porous nonwoven materials, open-celled foams, and apertured films. Examples include, without limitation, any flexible porous sheets of polyolefin fibers, such as polypropylene, polyethylene or polyester fibers; webs of spunbond polypropylene, polyethylene or polyester fibers; webs of rayon fibers; bonded carded webs of synthetic or natural fibers or combinations thereof. Either layer may also be an apertured plastic film. The various layers of the garment 2 have dimensions which vary depending on the size and shape of the wearer.

As seen in FIGS. 4-6, the garment 2 according to the present invention desirably will have the flaps 50, hereinafter described in the singular, extending in a long axis, or direction, corresponding to the longitudinal direction 62 of the garment 2. Other gasketing arrangements of additional areas may of course be desirable and are intended to fall within the scope of the present invention. The flap 50 will have an attached edge 52 attached, i.e. affixed to, or integral with, the garment, and a free edge 64 for contacting the body of the wearer 5. The flap 50 will have a transverse direction 66 perpendicular to its long direction 62. Arrows 68 indicate the long axis tensioning force achieved through addition of elastics 70 extending in the long direction of the flap 50. As seen in FIG. 5, the gasket 50, in its functional position, extends largely perpendicularly to the chassis 3. Extension of the gasket is indicated by dotted, or phantom, portion 65. Elastic members 70, or elasticity, may be provided such as discussed above or in any known manner sufficient to provide a normalizing force adequate to extend the flap in the transverse direction 66.

Breathable films suitable for use in constructing a gasket according to the exemplary embodiment will desirably satisfy the criteria of being transversely extensible, i.e. easily extensible and with a low recovery or modulus of elasticity in a first direction, and readily stretched or accepting of elastics, e.g. Lycra (TM) strands or otherwise providing a tensioning force, in a second direction perpendicular to the first as discussed above. The material should further provide suitable liquid barrier properties to function as a gasket, as well as providing breathability. Many microporous film types such as filled, unfilled, stretched, unstretched, crushed, or combinations thereof, may be suitable for use with the present invention with or without additional nonwoven layers laminated thereto. Additionally a soft feel and other esthetic properties are desirable.

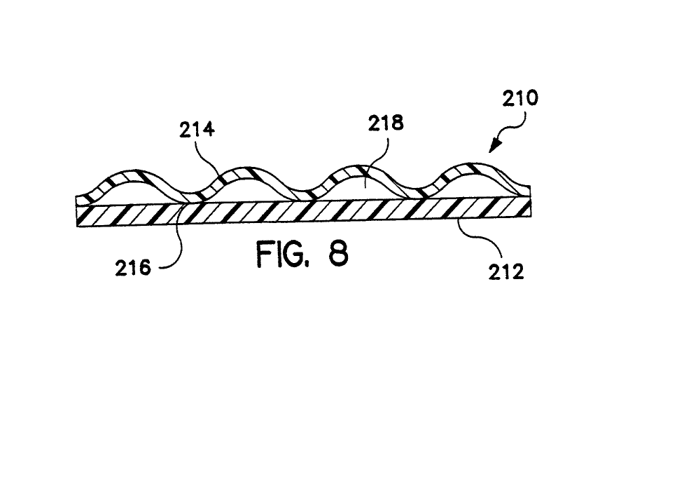

Referring to FIG. 8, a breathable liquid barrier composite or laminate 210 of the present invention in one desired form includes a microporous film layer 212 and a fibrous polyolefin nonwoven web comfort and support layer 214 which have been thermally bonded to one another. While this is a desirable configuration of the present invention, microporous film alone may be used, or additional layers of material may be added to composite 210 to form multilayered composites if so desired. For example, a second fibrous polyolefin nonwoven web (not shown) may be bonded to the film layer 212 on a side of the film opposite the first fibrous polyolefin nonwoven web 214.

The microporous film layer 212, and the breathable stretchable, laminate including the microporous film layer 212, may be produced generally according to the teachings of U.S. Pat. Nos. 5,695,868 and 5,855,999, both to McCormack, and incorporated by reference herein in their entirety. The person having ordinary skill in the art will appreciate that the specific examples of the breathable laminates therein will need to be modified to conform with the requirements of the present invention.

A variety of nonwoven web forming processes can be used with the present invention. Examples include, but are not limited to, air and wet laying, staple fiber carding and bonding, solution spinning, meltblowing and spunbonding processes. All of the foregoing processes are well known to those having ordinary skill in the art. Spunbond polypropylene webs work particularly well with the present invention. Spunbond webs can be made in accordance with the teachings of commonly assigned U.S. Pat. No. 4,340,563 to Appel. Spunbond materials are made by extruding molten thermoplastic material as filaments through a plurality of capillaries in a spinneret with the diameter of the extruded filaments than being reduced by, for example, eductive drawing or other well known spunbonding mechanisms. In some embodiments, bicomponent spunbond fibers, such as PRISM fibers, taught in U.S. Pat. No. 5,382,400, to Pike et al., may be used.

### MATERIAL EXAMPLES

The following material examples of breathable, liquid barrier, transversely stretchable materials are set forth by way of illustration for certain aspects of the invention are not intended to limit the scope or spirit of the present invention.

### Example 1

A breathable, stretchable, thin film laminate comprising spunbond facings of 0.6 osy polypropylene spunbond fibers, with a 0.55 osy core film of polyethylene, calcium carbonate and metallocene designated SCC22254, from Exxon, laminated to the spunbond facings, was tested according to the below listed test procedures and found to have a CD modulus of 48.16 psi/%, an MD modulus of 140.35 psi/%, an MD/CD Young's modulus ratio of 2.91, a hydrohead of 291.67 mbar, and a WVTR value of 11,200 gsm/24 hrs.

### Example 2

By way of comparison, a necked spunbond/film laminate comprising a single spunbond layer of a 0.4 osy layer of polypropylene wire weave spunbond fibers, and necked to about 45% of its original width, with a 1.25 osy polyether block amide PEBAX breathable elastomeric film from ATOFINA laminated to the spunbond layer, was tested according to the below listed test procedures and found to have a CD modulus of 13.87 psi/%, an MD modulus of 212.76 psi/%, an MD/CD Young's modulus ratio of 15.34, a hydrohead of at least 600 mbar and a WVTR value of at least 10,000 gsm/24 hrs.

Referencing especially FIG. 6, it is seen that an absorbent garment 2, upon becoming loaded with absorbent fluid, will sag under the force of gravity 77 away from the body of the wearer 5. This loss of vertical fit ordinarily creates a gap 79 between the gasket 50 and the wearer 5 leading to leakage. However, through provision of the flaps 50 of the exemplary embodiment, as garment 2 sags, the flap 50 extends toward the body 5 as indicated at line 81 in order to maintain contact with the body 5 thereby providing gasketing and preventing leakage to the exterior of the garment.

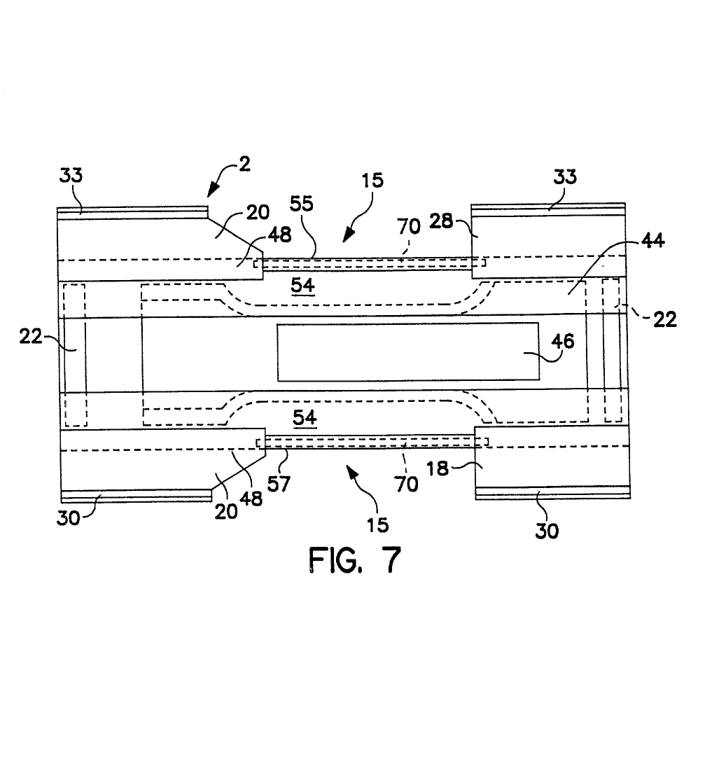

Referencing FIG. 7, in an embodiment wherein portions 54 of the outer covering 48 extend beyond the absorbent layer 44, the extended portions 54 can be made to serve as seamless leak guards. By “seamless,” it is meant that the leak guards are not separately attached and, thus, do not require a seam for attachment to the waste containment section 4. To effectively serve as leak guards, the difference in width between the absorbent layer and outer cover must be substantial, as opposed to trivial, in the central region 15 between the leg openings. Generally, the outer cover 48 is at least about 40% wider than the absorbent layer 44 in the central region 15. Desirably, the outer cover 48 is at least about 60% wider than the absorbent layer 44 in the central region 15. More desirably, outer cover 48 is at least about 80% wider, and most desirably at least about 100% wider than absorbent layer 44 in central region 15 on the underside of the garment. The outer cover 48 in this embodiment would be constructed and arranged from materials including elastics 70, selected according to the above discussed criteria including elastics 70, at least in so far as the gasketing area is concerned.

### Test Procedure for Water Vapor Transmission Rate (WVTR)

A suitable technique for determining the WVTR (water vapor transmission rate) value of a film or laminate material of the invention is the test procedure standardized by INDA (Association of the Nonwoven Fabrics Industry), number IST-70.4-99, entitled “STANDARD TEST METHOD FOR WATER VAPOR TRANSMISSION RATE THROUGH NONWOVEN AND PLASTIC FILM USING A GUARD FILM AND VAPOR PRESSURE SENSOR” which is incorporated by reference herein. The INDA procedure provides for the determination of WVTR, the permeance of the film to water vapor and, for homogeneous materials, water vapor permeability coefficient.

The INDA test method is well known and will not be set forth in detail herein. However, the test procedure is summarized as follows. A dry chamber is separated from a wet chamber of known temperature and humidity by a permanent guard film and the sample material to be tested. The purpose of the guard film is to define a definite air gap and to quiet or still the air in the air gap while the air gap is characterized. The dry chamber, guard film, and the wet chamber make up a diffusion cell in which the test film is sealed. The sample holder is known as the Permatran-W Model 100K manufactured by Mocon/Modem Controls, Inc., Minneapolis, Minn. A first test is made of the WVTR of the guard film and the air gap between an evaporator assembly that generates 100% relative humidity. Water vapor diffuses through the air gap and the guard film and then mixes with a dry gas flow which is proportional to water vapor concentration. The electrical signal is routed to a computer for processing. The computer calculates the transmission rate of the air gap and the guard film and stores the value for further use.

The transmission rate of the guard film and air gap is stored in the computer as CalC. The sample material is then sealed in the test cell. Again, water vapor diffuses through the air gap to the guard film and the test material and then mixes with a dry gas flow that sweeps the test material. Also, again, this mixture is carried to the vapor sensor. The computer than calculates the transmission rate of the combination of the air gap, the guard film, and the test material. This information is then used to calculate the transmission rate at which moisture is transmitted through the test material according to the equation:

TR⁻¹_{test material}=TR⁻¹_{test material, guardfilm, airgap}−TR⁻¹_{guardfilm, airgap}

Calculations:

WVTR: The calculation of the WVTR uses the formula:

WVTR=Fpₛₐₜ(T)RH/Apₛₐₜ(T)(1-RH))

where:

F=The flow of water vapor in cc/min.,

pₛₐₜ(T)=The density of water in saturated air at temperature T,

RH=The relative humidity at specified locations in the cell,

A=The cross sectional area of the cell, and,

pₛₐₜ(T)=The saturation vapor pressure of water vapor at temperature T.

Elongation Testing

A one inch strip of each material was evaluated on an Instron automated stress-strain tester. Specifically, the gap size between clamps on each side of the material during the stress-strain test was set at 0.25 inches. A Cross-head, or clamp separation, speed of 20 in/min was used. A maximum elongation of 200%, i.e. specifically from 1/4 inch to 3/4 inch where samples did not break. A maximum load: of 30 pounds was permitted. This procedure was used to measure the CD as well as the MD strength of the materials.

Hydrohead Testing:

In this test, water pressure is measured to determine how much water pressure is required to induce leakage in three separate areas of a test material. The water pressure is reported in millibars (mbars) at the first sign of leakage in three separate areas of the test specimen. The pressure in millibars can be converted to hydrostatic head height in inches of water by multiplying millibars by 0.402. Pressure measured in terms of inches refers to pressure exerted by a number of inches of water. Hydrostatic pressure is pressure exerted by water at rest.

Apparatus used to carry out the procedure includes a hydrostatic head tester, such as TEXTEST FX-3000 available from ATI Advanced Testing Instruments Corp. of Spartenburg, S.C., a 25.7 cm² test head such as part number FX3000-26 also available from ATI Advanced Testing Instruments Corp., purified water such as distilled, deionized, or purified by reverse osmosis, a stopwatch accurate to 0.1 second, a one-inch circular level, and a cutting device, such as scissors, a paper cutter, or a die-cutter.

Prior to carrying out this procedure, any calibration routines recommended by manufacturers of the apparatus being used should be performed. Using the cutting device, the specimen is cut to the appropriate size. Each specimen has a minimum size that is sufficient to allow material to extend beyond the outer diameter of the test head. For example, the 25.7 cm² test head requires a 6-inch by 6-inch, or 6-inch diameter specimen. Specimens should be free of unusual holes, tears, folds, wrinkles, or other distortions.

First, make sure the hydrostatic head tester is level. Close the drain faucet at the front of the instrument and pull the upper test head clamp to the left side of the instrument. Pour approximately 0.5 liter of purified water into the test head until the head is filled to the rim. Push the upper test head clamp back onto the dovetail and make sure the plug is inserted into the socket at the left side of the instrument. Turn the instrument on and allow the sensor to stabilize for 15 minutes. Make sure the Pressure Gradient thumbwheel switch is set to 60 mbar/min. Make sure the drain faucet is closed. The water temperature should be maintained at about 750 Fahrenheit ±10° Fahrenheit. Use the Light Intensity adjustment to set the test head illumination for best visibility of water droplets passing through the specimen.

Once the set-up is complete, slide the specimen onto the surface of the water in the test head, from the front side of the tester. Make sure there are no air bubbles under the specimen and that the specimen extends beyond the outer diameter of the test head on all sides. If the upper test head clamp was removed for loading the specimen, push the clamp back onto the dovetail. Pull down the lever to clamp the specimen to the test head and push the lever until it comes to a stop. Press the Reset button to reset the pressure sensor to ZERO. Press the Start/Pause button to start the test. Observe the specimen surface and watch for water passing through the specimen. When water droplets form in three separate areas of the specimen, the test is complete. Any drops that form within approximately 0.13 inch (3.25 mm) of the edge of the clamp should be ignored. If numerous drops or a leak forms at the edge of the clamp, repeat the test with another specimen. Once the test is complete, read the water pressure from the display and record. Press the Reset button to release the pressure from the specimen for removal. Repeat procedure for desired number of specimen repeats.

While the embodiments of the invention described herein are presently considered desirable, various modifications and improvements can be made without departing from the spirit and scope of the invention. The scope of the invention is indicated by the appended claims, and all changes that fall within the meaning and range of equivalents are intended to be embraced therein.

We claim:

1. An breathable, liquid impervious material suitable for a containment flap in an absorbent article.

2. The material of claim 3 wherein the material has a WVTR value of greater than about 5,000 gsm/24 hrs.

3. The material of claim 1 wherein the material is a laminate of thermoplastic film and nonwoven facing materials.

4. The material of claim 3 wherein the nonwoven facing material is a polypropylene spunbond.

5. The material of claim 3 wherein the material is a laminate of a layer of nonwoven facing material of about 0.4 osy basis weight and necked to about 45% of its original width and including spunbond substantially continuous polypropylene fibers and a layer of about a 1.25 osy basis weight polyether block amide film.

6. The material of claim 1 wherein the material comprises a microporous film.

7. The material of claim 1 wherein:
a) the material has a first axis and a second axis, and
b) the material has a Young's modulus of up to about 14 psi/% in the first axis.

8. The material of claim 7 wherein the material has a Young's modulus of up to about 212 psi/% in the second axis.

9. An absorbent article comprising:
a) an absorbent chassis, the chassis having a longitudinal axis;
b) a containment flap comprising a breathable liquid impervious barrier material, the flap having a free edge and an attached edge, the attached edge being attached to the chassis.

10. The absorbent article according to claim 9 wherein the barrier material comprises a microporous film.

11. The absorbent article of claim 9 wherein the containment flap comprises a transversely extendible film.

12. The absorbent article of claim 11 wherein the containment flap has a long axis and a transverse axis, the long axis being parallel to the longitudinal axis of the chassis, the flap having a tensioning force in its long axis, the flap having a low modulus of elasticity in its transverse axis and being extendible in its transverse axis.

13. The absorbent article of claim 12 wherein the modulus of elasticity is about 14 psi/% or lower.

14. The absorbent article of claim 12 wherein the tensioning force is sufficient to produce extension of the flap in the transverse direction.

15. The absorbent article of claim 9 wherein the flap is integral with an outer cover of the article.

16. The absorbent article of claim 9 wherein the flap includes elastics within the flap to supply the tensioning force.

17. The absorbent article of claim 11 wherein the transversely extendible film is a microporous film of about 10 to about 68 weight percent predominately linear polyolefin polymer about 2 to about 20 weight percent of a bonding agent, and about 30 to about 80 weight percent particulate filler.

18. The absorbent article of claim 17 wherein the polyolefin polymer is a linear low density polyethylene.

19. The absorbent article of claim 11 wherein the microporous film comprises a filler and first and second polymers, the first polymer being a blend of ethylene and propylene.

20. The absorbent article of claim 9 wherein the containment flap comprises a spunbond material.

21. The absorbent article of claim 20 wherein the spunbond material is a polyolefin.

22. The absorbent article of claim 21 wherein the spunbond material is polypropylene.

23. The absorbent article of claim 9 wherein the flap comprises crimped nonwoven/extensible film laminates.

24. The absorbent garment of claim 23 wherein the film comprises a stretched microporous film.

25. The absorbent article of claim 9, comprising one of a diaper; a training pant; an article of swim wear; an absorbent underpant; an adult incontinence article; a feminine hygiene article; or a medical protective garment.
